(19) United States
(12) Patent Application Publication
(10) Pub. No.: US 2008/0112983 A1
(43) Pub. Date: May 15, 2008

(54) Barn Dust Extract Providing Protection From Allergies

(75) Inventors: Albrecht Bufe, Hamburg (DE); Otto Holst, Bad Oldesloe (DE); Erika Von Mutius, Leutstetten/Starnberg (DE); Charlotte Braun-Fahrlander, Basel (CH); Dennis Nowak, Krailling (DE); Josef Riedler, Bischofshofen (AT)

Correspondence Address: KNOBBE MARTENS OLSON & BEAR LLP, 2040 MAIN STREET, FOURTEENTH FLOOR, IRVINE, CA, 92614, US

(73) Assignee: Albrecht Bufe, Hamburg (DE)

(21) Appl. No.: 11/575,490

(22) PCT Filed: Aug. 18, 2005
(86) PCT No.: PCT/EP05/08937

§ 371 Date: Oct. 31, 2007

(30) Foreign Application Priority Data
Sep. 18, 2004 (EP) 04022257.4

(51) Int. Cl. A61K 39/35; A61P 27/14; A61P 37/08; A61P 11/06

(52) U.S. Cl. 424/275.1

(57) ABSTRACT

The present invention describes a process for preparing an extract for the treatment of allergic diseases, in which process no heat is supplied during all of the preparation process, an extract which has been prepared by this process, and the use of this extract for preparing a medicament.

The present invention relates to a composition which contains all of the allergy-protecting potency, activity and effect of a natural barn dust, and a process for the preparation of this composition.

Excessive reactions of the body, in particular of the immune system, to nonhazardous foreign substances are referred to as allergies. These reactions proceed just as does the normal immune response to a pathogen. One distinguishes between several types of allergic immune responses. Allergic type I reactions, which include bronchial asthma, atopic dermatitis, urticaria, hay fever and food allergies, are most widespread. Although these diseases differ with regard to their physical symptoms, they are based on similar immunological mechanisms.

A large number of studies confirm that allergic diseases are on the increase. The causes for the incidence and development of allergies are as yet unclear. It is probable that certain, as yet unknown hereditary factors and environmental conditions such as exposure to allergens, city life, number of siblings and certain infections, are contributory factors.

Type I allergies are caused by group E antigens, whose development depends on group II T-helper lymphocytes. In allergies, these Th2 cells are more frequent relative to the Th1 cells. Children are first born with a Th2-dominated immune response, which probably as the result of the microbial load, shifts within their first year towards a Th1-dominated response (nonallergic immune response). It is assumed that frequent infections correlate with a lower Th2-response and thus with fewer allergies. Therefore, a low microbial load might lead to an increased incidence of allergies (hygiene hypothesis). A series of epidemiological studies in Europe have demonstrated that children who are raised on farms are much less frequently affected by allergic diseases than children who grow up under nontraditional environmental conditions (peasant effect). Presently, it is clear that this effect is due to the frequent contact with barns which children have who are raised on farms since the most pronounced protection against allergies was found in children who often frequented barns during the first year of their lives. This protective effect is most probably due to the contact with barn dust (for example via inhalation). The reason is that barn dust contains, inter alia, immunostimulatory active substances from bacteria, viruses, fungi, plants and animals, which are probably responsible for the peasant effect, which goes hand in hand with the hygiene hypothesis. However, the substances in the barn dust which ultimately trigger the protective effect are as yet unknown.

The possibilities of preventing and curing allergic diseases are limited. While the symptoms can be alleviated relatively well by a large number of medicaments, the success rates of immunotherapeutic treatments such as desensitization differ. This therapy is rather ineffective especially in the case of bronchial asthma, a frequent affliction. Avoiding allergens by way of preventative treatment will not certainly contribute to lowering the allergy frequency. Overall, reducing the risk of allergies developing is an impossibility.

WO 01/49319 describes a composition which contains antigens located on/in microorganisms and whose use is intended to be used for the prevention and treatment of allergic diseases. The composition is prepared by collecting barn dust and, if appropriate, suspending it in a suitable solvent, for example water or isotonic saline. This gives a suspension of microorganisms or else fragments of these organisms in a solvent, which suspension can be administered directly or after further processing steps for treatment of allergies.

WO 96/00579 describes a preparative process for the preparation of a suspension and an extract of microbacteria in aqueous solution, where the extract is heated for at least 20 minutes at 121° C. This suspension, or this extract, can be employed for the unspecific immune modulation.

It was an object of the present invention to provide a process for the preparation of a composition and a composition for the prevention and treatment of allergic diseases.

This object is achieved by a process for the preparation of an antiallergenic extract, characterized in that no heat is supplied during all of the preparation process, and an extract prepared by such a process.

The extract is preferably prepared from dusts which may comprise fungal and bacterial spores, but no vegetative cells. The extract is preferably prepared with barn dust from farms.

The dust can be collected with any type of collection system, i.e. by sweeping up, by aspiration, by wiping and the like, without the invention being limited thereby.

After collection, the dust may be homogenized. A suitable type of technique is any technique which leads to a uniform homogenization of the dust, in particular one which removes agglomerations and lumps of the dust. Suitable for these purposes are methods such as rubbing, smashing or crushing, stirring or introducing into a blender, without being limited thereto. Digesting the dust may also be part of the process according to the invention. Digesting of the constituents present in the dust, such as cells, microorganisms, in particular their spores and the like, can be effected, for example by grinding, squashing and similar methods.

In accordance with the invention, the extraction of the dust is preferably accomplished with the aid of water or of an aqueous solution, with a physiological aqueous saline or an aqueous buffer being especially preferred. A preferred aqueous buffer contains sodium salts or similar mono- or divalent salts such as Na₂SO₄, KCl, LiCl, MgCl₂ and CaCl₂. Accomplishing the extraction of the dust with a physiological saline (0.9% NaCl) is very especially preferred.

It is an important aspect of the process according to the invention that no external heat is supplied during all of the processing procedure of the extract preparation. This means that neither the solvents employed are heated (at the point in time of use) nor is heat supplied during the preparation process. Rather, the extract is prepared at ambient temperature or below. This means that all solvents used are at ambient temperature (between approx. 10° C. and 40° C., depending on the season and the place of preparation) or may be cooled, but are not used while hot, nor is the extract heated.

After the dust has been taken up in the aqueous solution or in water, this suspension is either left to stand or stirred, so that the water-soluble substances, or the substances which can be removed from the dusts by water, may enter into the aqueous phase. Thereafter, the dust particles which remain in the solution as solid components are removed by a sedimentation step. The sedimentation step is not limiting for the process according to the invention; any type of sedimentation which is known to the skilled worker may be employed. Also, leaving the suspension to stand so that sedimentation of the solid constituents can be accomplished by the earth's gravity may be considered as being for the purposes of the invention. A preferred way of removing the solid constituents is centrifuging, whereafter the supernatant is removed from the sediment. The supernatant, from which the solid constituents such as cells, microorganisms or spores have been removed from the dusts by means of sedimentation, forms the extract according to the invention.

In a preferred embodiment of the invention, the extract can optionally also be subjected to a dialysis step. The dialysis may be accomplished against the same type of solvent as have already been employed for taking up the dust. Preferably, the dialysis is accomplished against distilled water. A preferred exclusion limit for the dialysis is a limit of approx. 3000 Daltons. Thus, smaller molecules are removed from the extract.

An extract prepared by this process is distinguished in terms of quality by having, with equal amounts of dust employed and equal amounts of solvents employed, a markedly higher protein content than extracts which are prepared by traditional extraction methods; moreover, the protein distribution (the protein pattern on an SDS gel) can be distinguished from that of other extracts. Moreover, the extract is distinguished by the fact that it has a pronounced saponin content.

The extract according to the invention can be used for the prevention and treatment of allergic diseases; in particular, it can be used as medicament, or for the preparation of a medicament, for the prevention and treatment of diseases caused by allergies, such as bronchial asthma, hay fever, atopic dermatitis, food allergies, urticaria and all types of contact allergies. The efficacy of the extract according to the invention markedly exceeds the efficacy of extracts which are prepared by traditional processes, as is shown in the examples.

### FIGURES

FIG. 1 shows the separation profile of the separation of the composition according to the invention and of three comparative extracts by means of HPLC.

FIG. 2 shows the separation of the composition according to the invention and three comparative extracts by means of SDS-PAGE. The gel at the top has been stained with Coomassie Blue, while the gel at the bottom shows a silver staining of the same separation.

FIG. 3 shows a diagrammatic resume of the asthma and sensitization model in Balb/c laboratory mice. The mice are administered the allergen (ovalbumin grade VII) intraperitoneally twice with a 14-day-interval, and then twice in the form of an aerosol. In parallel, the mice, which are kept in a sealed chamber, inhale the composition according to the invention 20 minutes per day over a period of 14 days. On day 39, the respiratory function of the lungs is measured with a body plethysmograph for mice; on day 41, the remaining serological and cell-biological analyses follow.

FIG. 4 shows the results of studies of asthma symptoms in mice. AUC means “area under the curve” and describes the area under the Penh curve after the mice have been stimulated with 6, 12, 24 and 48 mg/ml metacholin. The comparison is between mice which have inhaled PBS as negative control (PBS), LPS in barn dust equivalent dose as negative control (LPS), the composition according to the invention (NaCl_{cold}), a cold-water extract by way of comparison (H₂O_{cold}), the methanol phase of the Bligh & Dyer extract (CHCl₃/MeOH), and PBS without sensitization (non sens.).

FIG. 5 shows the modulation of the eosinophilic cells in bronchoalveolar lavage (BAL). The comparison is between mice which have inhaled PBS as negative control (PBS), LPS in barn dust equivalent dose as negative control (LPS), the composition according to the invention (NaCl_{cold}), a cold-water extract by way of comparison (H₂O_{cold}), the methanol phase of the Bligh & Dyer extract (CHCl₃/MeOH), and PBS without sensitization (non sens.).

### EXAMPLES

### Example 1

### Collection of Dust from Cattle Houses and Barns

The sediment dust collection is preferably made in cow and calf houses and in barn areas of traditionally managed Alpine farms. The latter are defined via their position in the northern Alpine valleys in Switzerland, Austria and Germany, and by their particular architecture, i.e. the spatial vicinity of animal houses and living quarters. The sediment dust is sampled from the frames of barn gates, windowsills, raised shelving, cow-trainer bars, shelves, stalls, raised shelvings and scattered utensils. The height of the collection surfaces should be between 0.5 m and 1.5 m above ground. No moist dust, in particular no wet dust, must be collected. The surfaces are scraped with a metal spatula and sieved through a domestic sieve (ALK-Scherax filter attachment [prefractionator sieve] for vacuum cleaners) into the dust collection container for barn dust.

All of the collected samples are stored dry in the sealed collection container at room temperature (22° C. maximum) and protected from direct sunlight.

### Example 2

### Extraction

The barn dust is homogenized with a spatula and then in a mortar. Thereafter, the dust homogenized in this way is suspended in 0.9% aqueous NaCl solution (at 22° C.), disrupted at approx. 4° C. using glass beads and stirred carefully for a further 6 hours at 220C. Thereafter, the mixture is centrifuged, the supernatant is removed, dialyzed against water and lyophilized. The yield of the NaCl_{cold} extract (composition according to the invention) amounts to 9% by weight of the barn dust employed. For comparison, barn dust extracts were prepared with H₂O_{cold} (16.8% by weight), H₂Oₕₒₜ (20.2% by weight) and CHCl₃/MeOH (9% by weight).

### Example 3

### Biochemical Characterization

The composition according to the invention is subjected to a fingerprint analysis by means of HPLC in comparison with the three other extracts and characterized by SDS-polyacrylamide gel electrophoresis (SDS-PAGE) for the presence of proteins (Coomassie staining, silver staining) and lipoglycans (silver staining). Sugars, protein (total content) and fatty acids are also determined quantitatively. Standardized routine methods are employed for this purpose.

Characterization of the Extracts by HPLC and SDS-PAGE:

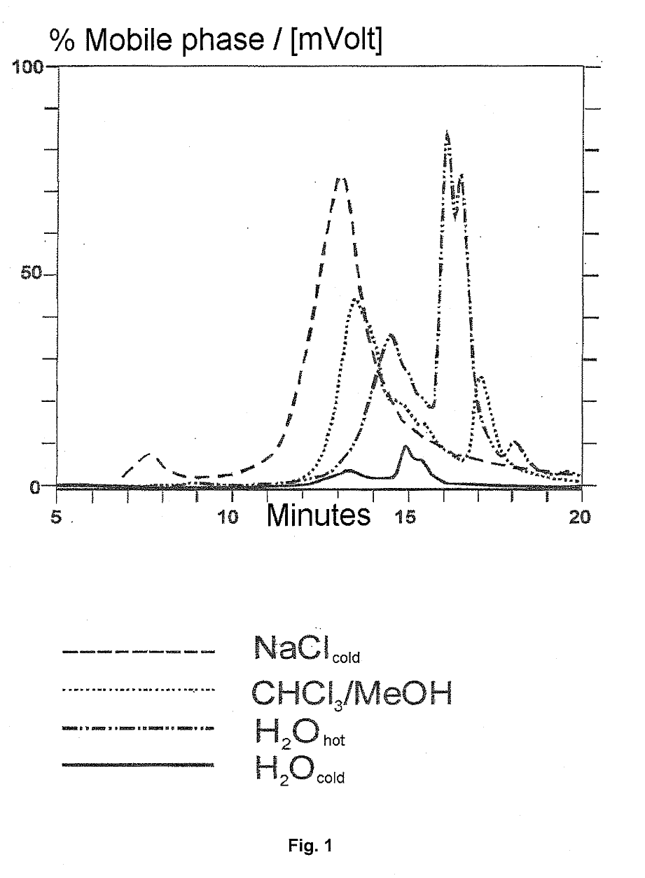

For the HPLC characterization, the extracts are separated by a Zorbax Bioseries GF-250 column (0.2 M Na phosphate buffer, pH 7.0, isocratic, protein separation by size, detection at 280 nm). In comparison with the other dust extracts, a characteristic separation profile for the composition according to the invention can be identified at 280 nm; this is shown in FIG. 1.

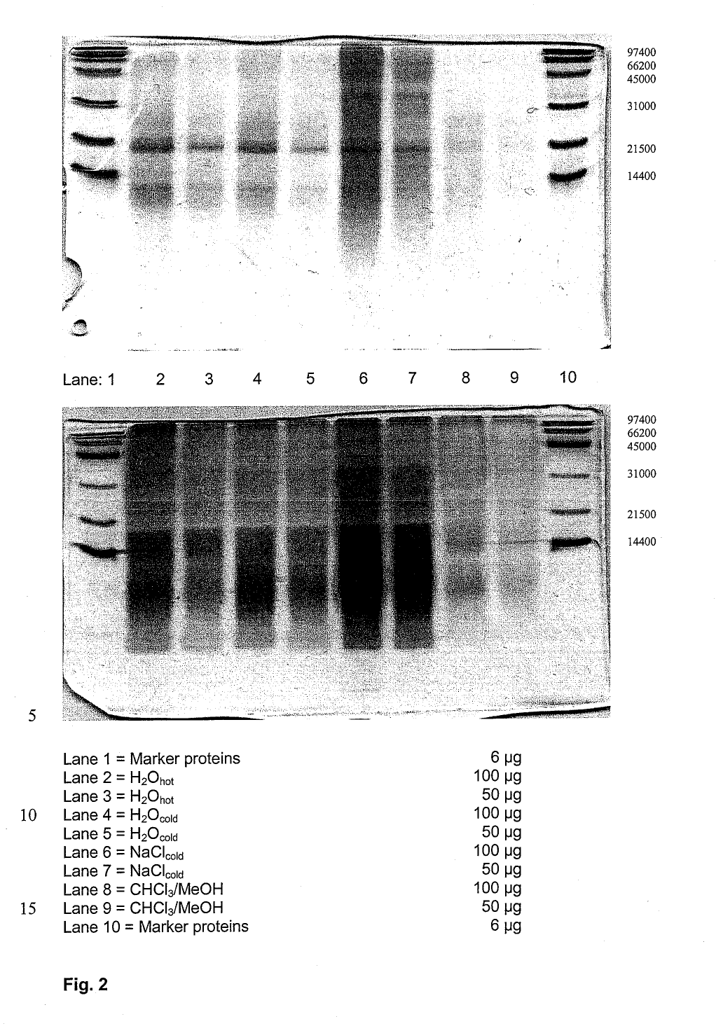

The separation of the extracts in the SDS-PAGE (FIG. 2) shows that the composition according to the invention differs from the other extracts in terms of both quality and quantity. This applies both to proteins (Coomassie staining, top image: MW 30 000-100 000 Da) and lipoglycans (silver staining: broad bands produced by (a) substance(s) in the MW range 20000 and below). In addition, the silver staining reveals further, distinct bands which are only very weakly stained by Coomassie, or not at all (MW approx. 26 kDa, approx. 14 kDa, approx. 12 kDa and 2 bands below). These may be proteins and/or glycoproteins which are only very weakly stained by Coomassie (strongly acidic proteins) and/or which are only present in small amounts (silver staining is between 10 and 40 times more sensitive than Coomassie staining). In particular, the extract according to the invention contains markedly more proteins with high molecular weights than the comparative extracts, a protein with approx. 20 kDa being particularly present in all extracts.

At approx. 140 μg/mg, the protein content of the composition according to the invention is twice as high than the protein quantities of the other extracts when identical starting materials of barn dust were used and the same volumes were employed in the process.

The analysis of the sugar composition demonstrated that none of the extracts contain any uronic acids and heptoses and that all extracts contain glucosamine and galactosamine. As regards the neutral sugars, 34.6 nmol/mg of rhamnose, 20.9 nmol/mg of ribose, 42.0 nmol/mg of arabinose, 25.6 nmol/mg of xylose, 70.9 nmol/mg of mannose, 129.7 nmol/mg of glucose and 124.4 nmol/mg of galactose are found in the composition according to the invention.

TABLE 1
Sugar determination of dust extracts (nmol/mg dust extract)

| Samples | Anal. | Rha | Rib | Ara | Xyl | Man | Glc | Gal | Hep | GlcA | GalA | GlcN | GalN |
|---|---|---|---|---|---|---|---|---|---|---|---|---|---|
| H₂Oₕₒₜ | NS | 23.89 | 15.59 | 88.05 | 41.92 | 81.94 | 293.11 | 181.22 | 0 | | | | |
| | UA | | | | | | | | | 0 | 0 | | |
| | AS | | | | | | | | | | | yes | yes |
| H₂O_{cold} | NS | 20.80 | 0 | 51.50 | 29.67 | 83.06 | 111.45 | 132.50 | 0 | | | | |
| | UA | | | | | | | | | 0 | 0 | | |
| | AS | | | | | | | | | | | yes | yes |
| NaCl_{cold} | NS | 34.60 | 20.89 | 42.04 | 25.62 | 70.91 | 129.65 | 124.37 | 0 | | | | |
| | UA | | | | | | | | | 0 | 0 | | |
| | AS | | | | | | | | | | | yes | yes |
| CHCl₃/MeOH | NS | 99.99 | 0 | 0 | 25.74 | 76.04 | 317.78 | 143.21 | 0 | | | | |
| | UA | | | | | | | | | 0 | 0 | | |
| | AS | | | | | | | | | | | yes | yes |

Anal. = analytical method;
NS = neutral sugars;
UA = uronic acids;
AS = amino sugars;
Rha = rhamnose;
Rib = ribose;
Ara = arabinose;
Xyl = xylose;
Man = mannose;
Glc = glucose;
Gal = galactose;
Hep = heptose;
GlcA = glucuronic acid;
GalA = galacturonic acid;
GlcN = glucosamine;
GalN = galactosamine

As can be seen from table 1, H₂Oₕₒₜ contains markedly higher amounts of arabinose, xylose, glucose and galactose, and the CHCl₃/MeOH extract contains higher amounts of rhamnose and glucose. The neutral sugar composition of H₂O_{cold} does not differ substantially from that of the composition according to the invention. These findings suggest that the extraction temperature is an essential contributory factor for molecular differences.

The fatty acid analysis reveals the following contents for the composition according to the invention: C12:0-5.9 nmol/mg, C14:0-30.7 nmol/mg, 2-OH—C14:0-4.3 nmol/mg, 3-OH—C14:0-3.2 nmol/mg, C16:0-13.9 nmol/mg, C18:0-5.6 nmol/mg and C18:1-4.7 nmol/mg. In total, this means 68.2 nmol/mg fatty acids.

TABLE 2
Total fatty acid determination of dust extracts (nmol FA/mg dust extract)

| Samples | C12:0 | C14:0 | 2OH—C14:0 | 3OH—C14:0 | C16:0 | C18:0 | C18:1 | Total |
|---|---|---|---|---|---|---|---|---|
| H₂Oₕₒₜ | 5.07 | 18.53 | 2.51 | 2.95 | 9.12 | 4.03 | 3.94 | 46.15 |
| H₂O_{cold} | 2.93 | 14.21 | 1.34 | 2.95 | 9.43 | 4.12 | 2.89 | 37.87 |
| NaCl_{cold} | 5.88 | 30.69 | 4.32 | 3.17 | 13.88 | 5.55 | 4.70 | 68.19 |
| CHCl₃/MeOH | 4.93 | 15.98 | 4.37 | 4.03 | 14.56 | 4.63 | 1.75 | 50.25 |

As can be seen from table 2, the composition according to the invention has a markedly higher C14:0 and C16:0 content than the two other aqueous extracts. These results demonstrate that not only the temperature, but also the addition of NaCl results in the extraction of different molecules from barn dust.

### Example 4

### In-Vivo Efficacy

The overall protective potency of the composition according to the invention is determined in vivo by immunological and cell-biological function studies in the standardized animal model of the mouse.

The following text will first describe the methods for sensitization and induction of allergic asthma in mice with ovalbumin, the treatment with barn-dust extracts and the analysis of the allergic and inflammatory reactions.

Sensitization and induction of allergic asthma in mice with ovalbumin (Ova)—Aluminum hydroxide (Pierce) is used as the adjuvant for sensitizing mice against the model allergen ovalbumin. This adjuvant is known to trigger immune responses which are characterized by the production of cytokins such as IL-4, IL-5 and IL-13 and by the production of antibodies of the IgE and IgG1 isotype (corresponding isotype in humans: IgG4).

Seven-week-old mice of the inbred strain Balb/c are sensitized by intraperitoneal injection of a mixture of 20 μg of ovalbumin (grade V from Sigma) and 2.2 mg of aluminum hydroxide in a total volume of 200 μl of buffer (PBS). In total, the injection is administered twice, i.e. on day 0 and on day 14.

To subsequently trigger an allergic reaction in the mice's lungs, i.e. to trigger the acute allergic asthma, the mice are treated in each case for 20 minutes with ovalbumin aerosol on day 28 and day 38. To this end, the mice are placed into a Plexiglas chamber with a volume of 11 liters. The chamber is connected with an inhaler from Pari which had previously been filled with 1% strength ovalbumin solution and which is operated by a Pari-Boy-Turbo aerosol generator. After the second application of OVA aerosol, acute allergic asthma symptoms can be diagnosed in the mice over a few days. The symptoms include:
1) the increased response to methacholin, the bronchial hyperreactivity
2) the increased infiltration of eosinophilic granulocytes and lymphocytes into the respiratory lumen
3) the production of ovalbumin-specific antibodies of the IgE and IgG1 isotypes
4) the production of an interleukin-5 after in-vitro restimulation of lymphocytes with ovalbumin

The methods for measuring the individual parameters are illustrated in greater detail hereinbelow.

Measurement of the bronchial hyperreactivity—A decisive diagnostic parameter which is measured in humans in order to reliably diagnose an allergic asthmatic disease is the bronchial hyperreactivity. This is a hypersensitivity of the smooth bronchial muscles to unspecific stimulation. For the diagnosis in humans, low histamine concentrations are administered, and the respiratory resistance is then measured in a plethysmograph. Asthma sufferers show a more pronounced bronchoconstriction as a function of the histamine concentration, and thus a higher respiratory resistance than healthy subjects.

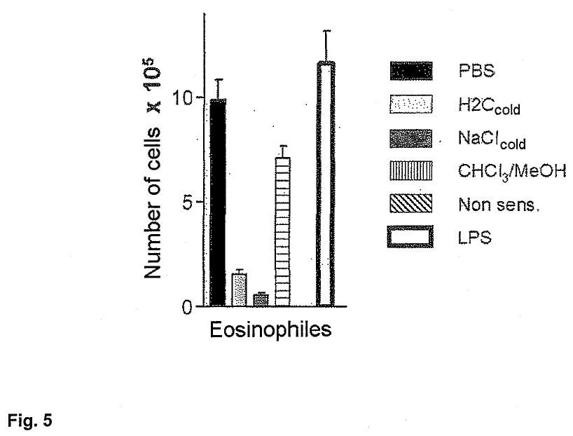

In mice, the measurement is very similar. For the measurement, the mice are introduced into a plethysmograph from Buxco in the chambers provided. A continuous stream of air flows through the chambers, and a highly sensitive pressure gauge registers short-term pressure changes in the chamber. The mice's aspiration generates a lower pressure in the chamber and their expiration a higher pressure. This pressure change is continuously registered as a function of time, and the values measured are used by the software to calculate the so-called Penh value for each breathing cycle. The formula by means of which the Penh value is calculated can be seen from FIG. 5. This dimension-free value correlates with the respiratory resistance. In hyperreactive mice, the respiratory resistance increases as a function of the methacholin concentration (in mice, methacholin is used for the provocation in place of histamine since the bronchi of mice do not react to histamine), which is expressed by an increased Penh value.

The bronchial hyperreactivity is measured 24 hours after the last exposure to OVA aerosol. To measure the bronchial hyperreactivity, the mice are allowed to acclimatize for 10 minutes in the plethysmograph and are then brought into contact with PBS aerosol for 7 minutes, during which process the Penh value is recorded. They are then exposed for in each case 7 minutes to increasing methacholin concentrations: 6, 12, 25, 50 mg/ml. During each exposure, the Penh value is measured continuously and averaged over 30-second periods. The period within which the maximum Penh value is reached for a certain methacholin concentration is then plotted versus the methacholin concentration. This results in a dose-effect curve, whose area under the curve increases with an increasing reaction, by the mice, to methacholin. The area under the curve thus correlates with the animals' hyperreactivity, and is used in the statistical evaluation.

Studying the cellular composition of the broncho-alveolar ravage (BAL)—In healthy subjects or untreated mice, only very few, if any, eosinophilic granulocytes can be observed in the lung tissue and in the lumen of the respiratory pathway. The leukocytes found in healthy respiratory pathways are mostly macrophages. In connection with allergic asthma, a massive infiltration of the respiratory pathways by eosinophilic granulocytes, and, to a lesser extent, by lymphocytes, takes place, the reasons for which are not entirely clear.

To study the composition of the leukocyte infiltrate in the respiratory pathways of mice, the animals are sacrificed three days after the last exposure to OVA aerosol. The windpipe is exposed, and a 24 gauge butterfly needle is inserted into the windpipe. The lumen of the respiratory pathways is rinsed twice through this needle, using in each case 1 ml of isotonic buffer (broncho-alveolar lavage). The total number of cells washed from the lung in this manner is counted under the microscope using a Neubauer hematocytometer. Thereafter, the leukocytes obtained by the BAL are centrifuged onto slides using a Zyto centrifuge from Shandon. Then, the cells are stained with the HAEME rapid staining kit from Labor+Technik Eberhard Lehmann as described in the package insert. After the staining, 300 cells on the slide are counted under the microscope and assigned to the different leukocyte subpopulations, following the customary criteria. Thereafter, the percentage composition of the BAL can be calculated.

Measurement of OVA-specific antibodies of isotypes IgE and IgG1—The sensitization to an allergen can be measured by determining allergen-specific antibodies of the classes IgG1 and IgE. The allergen-specific antibodies in the serum of mice are determined quantitatively by means of indirect ELISA. To this end, blood is taken from the mice's tail vein two days after the last administration of OVA aerosol. After the blood has clotted, the serum is obtained by centrifugation and employed in the ELISA:

### Brief Instructions for ELISA:

### IgE (OVA-Specific) ELISA:

coat plates with 5 μg/ml OVA with coating buffer, 100 μl per well (overnight)
wash 3×, 250 μl per well
blocking with 1% BSA in wash buffer, 250 μl per well, 1 hour
wash 3×
apply serum dilutions in wash buffer 1/3 1/10 and 1/30, 100 μl per well, 1 hour
wash 3×
apply biotinylated anti-IgE antibody (Pharmingen, R35-72), diluted 1/100 in wash buffer, 100 μl per well, 1 hour
wash 3×
apply Extravidin-POD (Sigma), diluted 1/1000 in wash buffer, 100 μl per well, 1 hour
wash 3×
apply TMB substrate solution, 100 μl per well, 30 minutes
stop reaction with 50 μl 1 M sulfuric acid per well, plate can be read at a wavelength of 450 nm

### IgG1 (OVA-Specific) ELISA:

coat plates with 5 μg/ml OVA with coating buffer, 100 μl per well (overnight)
wash 3×, 250 μl per well
blocking with 5% MMP in wash buffer, 250 μl per well, 1 hour
wash 3×
apply serum dilutions in dilution buffer 1/100 1/300, 1/1000 and 1/3000, 100 μl per well, 1 hour
wash 3×
apply AKP conjugated anti-IgG1 antibody (Pharmingen, X56), diluted 1/1000 in dilution buffer, 100 μl per well, 1 hour
wash 3×
apply pNPP substrate solution, 100 μl per well, 30 minutes
stop reaction with 50 μl, 1 M sulfuric acid per well, plate can be read at a wavelength of 405 nm

A dilution series of a standard serum is included on each ELISA plate. This standard serum contains a known concentration of OVA-specific antibodies of the IgG1 and IgE isotypes. The concentration in the unknown sample can be calculated by comparing readings from murine serum with an unknown OVA-specific IgG1 and IgE concentration with the measuring series of the known murine serum.

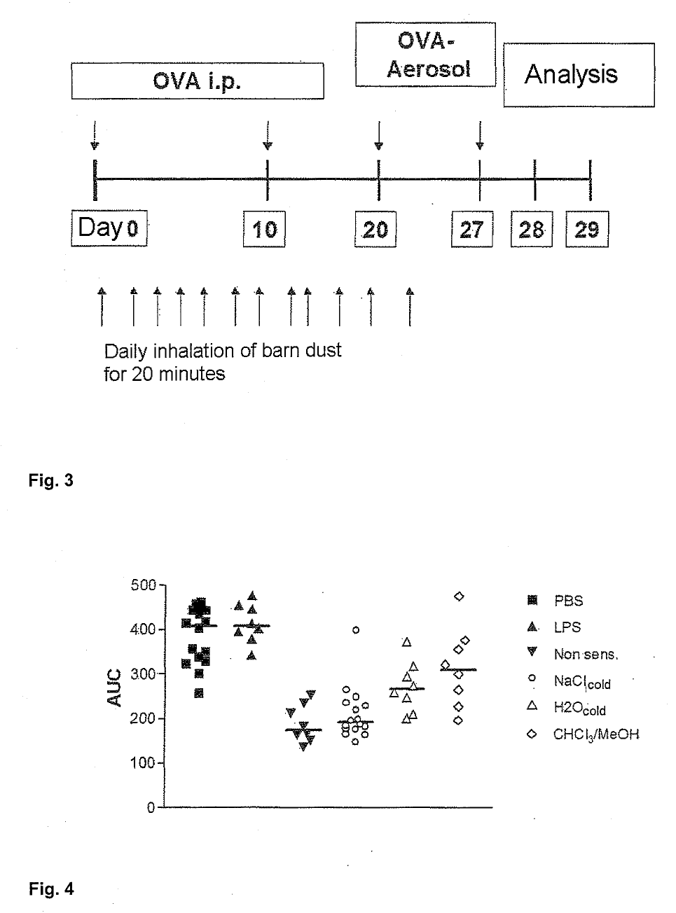

Treatment of mice with barn dust extract during the sensitization: To treat mice with the different barn dust extracts, the mice are placed into a Plexiglas chamber with a volume of 11 liters. The chamber is connected to a Pari-Boy aerosol generator as described above in the chapter “Sensitization and induction of allergic asthma in mice with ovalbumin (OVA)”, the inhaler previously having been filled with a 1% strength solution of the barn dust extracts. The mice are subsequently treated for 20 minutes with the barn dust extract aerosol. In total, the treatments are carried out 14 times, at the points in time, during the sensitization procedures, specified in FIG. 3.

### Example 5

### Results of the In Vivo Experiments

Asthma symptoms—the most comprehensive experiment for detecting the biological effect of the composition according to the invention is the study of the asthma symptoms in the mice which have been caused by allergy. FIG. 4 shows that the sensitivity of the respiratory passages in the asthmatic mice can be lowered to the level of untreated mice without asthma by treatment with the extract according to the invention. To rule out that the biological effect of the composition according to the invention is not caused by LPS, pure LPS was employed as control in a concentration as measured in the barn dust extract. LPS cannot reduce the mice's asthma symptoms. It can therefore be assumed that other substances in the dust are responsible for the biological effect which has been observed. CHCl₃/MeOH is the traditional extraction method by means of which, following phase separation, lipids are extracted into the chloroform phase and other molecules with weaker lipophilic properties are extracted into the methanol phase. The methanol extract (hereinbelow termed CHCl₃/MeOH) has no preventative effect on the mice's asthma symptoms. The barn dust extracted into H₂O_{cold} causes a partial reduction of the symptoms which, however, is not as pronounced as the effect of the composition according to the invention.

Inflammation in the bronchi—In mice with asthma, eosinophilic cells in the bronchi reflect the state of an asthmatic inflammation. FIG. 5 confirms that as the result of the treatment of the mice with the composition according to the invention, the number of eosinophilic cells, which are greatly increased in diseased mice and normally absent, drops rapidly and only few can be found. As in the case of the asthmatic symptoms, no change is observed in the mice which have been treated with buffer and LPS, CHCl₃/MeOH only has a minor effect, and the H₂O_{cold} extract falls somewhat short of the effect of the composition according to the invention.

Allergic sensitization—The asthma symptoms are triggered in the mice by sensitization with an allergen. A study was therefore carried out to find out whether the IgE antibodies against the allergen, whose number increases with sensitization, are influenced by the composition according to the invention. The table documents that the number of the IgE antibodies against ovalbumin only drops significantly in statistical terms in comparison with the untreated mice only when applying the composition according to the invention, while this number does not drop in the case of the other controls, or at least not significantly. While the increase in the IgG1 antibodies in the case of ovalbumin sensitization does not correlate with the asthma symptoms, it reflects a reaction of the T-lymphocytes of the Helper-2 type (Th2), whose increase can be observed in allergies. Here, the table shows that not only the composition according to the invention, but also the two other dust extracts can reduce the Th2 response of the immune system, but not LPS. The Th2 response of the immune system is therefore no precise marker for the particular biological effect of the composition according to the invention. Moreover, the active substances which are active in the composition according to the invention appear to be present to some extent in the other extracts, with the exception of the LPS solution.

TABLE 3
Effect of the various dust extracts on the level of the specific antibodies
against ovalbumin in the serum (IgE and IgG1), as measured by ELISA
and shown as relative units. The mice had inhaled the composition
according to the invention for 20 minutes per day over a 14-day period.

| Treatment | n | IgE (U/ml) ± SEM | IgG1 (U/ml) ± SEM |
|---|---|---|---|
| PBS | 16 | 4.4 × 10⁵ ± 1.1 × 10⁵ | 14 × 10⁵ ± 1.9 × 10⁵ |
| LPS | 8 | 2.8 × 10⁵ ± 0.7 × 10⁵ | 17.5 × 10⁵ ± 4.2 × 10⁵ |
| H₂O_{cold} | 8 | 4.6 × 10⁵ ± 1.4 × 10⁵ | 7.5 × 10⁵ ± 3.1 × 10⁵* |
| NaCl_{cold} | 16 | 1.9 × 10⁵ ± 0.4 × 10⁵* | 6.1 × 10⁵ ± 1.2 × 10⁵** |
| CHCl₃/MeOH | 8 | 1.7 × 10⁵ ± 1.0 × 10⁵ | 6.3 × 10⁵ ± 1.9 × 10⁵* |
| Non sens. | 4 | 0 | 0 |

*p < 0.05 versus PBS-treated mice;
**p < 0.01 versus PBS-treated mice

1. A process for the preparation of an antiallergenic extract from burn dust from farms, comprising extracting said extract with aid of water, a salt solution or a buffer solution wherein no heat is supplied during all of the preparation process.

2. (canceled)

3. (canceled)

4. The process as claimed in claim 1, wherein said salt solution is a physiological saline.

5. The process as claimed in claim 1, further comprising a step of sedimenting solid constituents.

6. The process as claimed in claim 1, further comprising performing a dialysis step.

7. The process as claimed in claim 1, comprising the following steps:
a) collecting barn dust,
b) homogenizing the dust,
c) suspending the homogenized dust in aqueous 0.9% strength NaCl solution at ambient temperature or below,
d) if appropriate disrupting the dust components,
e) sedimenting the solid constituents of the suspension thereby obtaining an aqueous supernatant, and
f) performing dialysis of the aqueous supernatant.

8. An extract prepared by a process as claimed in claim 1.

9. The extract as claimed in claim 8, wherein said extract contains saponins.

10. (canceled)
